(19) United States
(12) Patent Application Publication
(10) Pub. No.: US 2021/0002690 A1
(43) Pub. Date: Jan. 7, 2021

(54) COMPOSITIONS AND METHODS FOR DIAGNOSING URINARY TRACT INFECTIONS

(71) Applicant: Zomedica Pharmaceuticals Corp., Ann Arbor, MI (US)

(72) Inventors: Stephanie Morley, Ann Arbor, MI (US); Casey J. Wegner, Ann Arbor, MI (US)

(21) Appl. No.: 16/904,358

(22) Filed: Jun. 17, 2020

Related U.S. Application Data
(60) Provisional application No. 62/870,849, filed on Jul. 5, 2019.

(51) Int. Cl. C12Q 1/04; G01N 1/38; G01N 33/493; G01N 21/65; G06N 3/08; G06N 3/04; G16H 10/40; G16H 50/20

(52) U.S. Cl. CPC C12Q 1/04; G01N 1/38; G01N 33/493; G01N 21/65; G01N 2001/386; G06N 3/0454; G16H 10/40; G16H 50/20; G06N 3/08

(57) ABSTRACT

Provided herein are compositions and methods for diagnosing urinary tract infections. In particular, provided herein are compositions and methods for preparing canine urine samples and performing Raman spectroscopy detection of urinary tract infections in the samples.

This application claims priority to U.S. provisional patent application Ser. No. 62/870,849, filed Jul. 5, 2019, which is incorporated herein by reference in its entirety.

### FIELD OF THE DISCLOSURE

Provided herein are compositions and methods for diagnosing urinary tract infections. In particular, provided herein are compositions and methods for preparing canine urine samples and performing Raman spectroscopy detection of urinary tract infections in the samples.

### BACKGROUND OF THE DISCLOSURE

Urinary tract infection (UTI) is an infection caused by bacteria, fungi, or parasites in the urinary tract (kidneys, ureters, bladder & urethra). UTIs are common in pets and most common in dogs. UTIs may lead to increased frequency of urination, urgency, bloody urination and inappropriate urination in pets.

The infection is usually caused by bacteria in the environment or fecal contamination and subsequent spread of bacteria up the urinary tract and proliferation. Common bacteria involved include Staphylococcus, Streptococcus, Proteus, and E. coli. Crystals in urine may also contribute to infection and may be indicative of underlying disease/conditions. Leukocytes in urine can also be indicative of a UTI.

Proper diagnosis and treatment of UTIs in dogs typically requires culturing a urine sample to identify the causative bacteria. Such cultures take multiple days and may require sending a sample to an outside facility.

What is needed are rapid and efficient methods for detection of UTIs in dogs, preferably at the point of care.

### SUMMARY OF THE DISCLOSURE

Provided herein are compositions and methods for diagnosing urinary tract infections. In particular, provided herein are compositions and methods for preparing canine urine samples and performing Raman spectroscopy detection of urinary tract infections in the samples.

The compositions, systems, and methods of the present disclosure provide rapid, point of care detection of UTIs in canine samples. The detection methods provide multi-omic, multiplex detection without the need for costly and time-consuming reagents and complicated sample preparation. The described methods result in improved care of canine subjects with UTIs.

For example, in some embodiments, provided herein is a method of identifying the presence of a urinary tract infection (UTI) in a urine sample (e.g., from a canine), comprising: a) diluting the urine sample with water to generate a diluted sample; and b) obtaining a Raman spectrum of the diluted sample using a Raman spectrometer, wherein the Raman spectrum identifies the presence of a UTI in the sample. In some embodiments, the sample is diluted with purified or unpurified water (e.g., tap water, filtered water, sterilized water, distilled water, reverse osmosis water, or deionized water). In some embodiments, the sample is filtered prior to obtaining a Raman spectrum (e.g., with a polycarbonate membrane, a mixed cellulose ester, polyvinylidene difluoride, nylon, or cellulose acetate). In some embodiments, samples are filtered prior to dilution. In some embodiments, filtration and dilution replace centrifugation.

The present disclosure is not limited to a particular level of dilution of the sample. For example, in some embodiments, the sample is diluted at least 20%, 50%, 75%, 80%, 100%, 150%, 200%, or more). In some embodiments, the diluting step comprises i) centrifuging the urine sample to generate a pellet and a supernatant; and ii) removing at least a portion of the supernatant and replacing it with water to generate a diluted sample. In some embodiments, at least 20%, 50%, 75%, or 100% of the supernatant is replaced with water.

After sample preparation, samples are analyzed using Raman spectroscopy. The present disclosure is not limited to particular Raman spectrometers. In some embodiments, commercially available Raman spectrometry systems are utilized. (See also U.S. Pat. No. 10,253,346, and U.S. patent application Ser. No. 16/451,901; each of which is herein incorporated by reference in its entirety).

In some embodiments, the presence of a UTI is determined based on the presence of bacteria and/or crystals in the urine sample. In some embodiments, the diluted sample exhibits reduced or no background interference from urine. In some embodiments, the Raman spectroscopy generates a molecular fingerprint of the sample. In some embodiments, the molecular fingerprint comprises spectral bands indicative of a one or more of proteins, nucleic acids, carbohydrates, and small molecules (e.g., metabolites). In some embodiments, the molecular fingerprint identifies the genus, species, or strain of bacteria in the sample and the presence or type of crystals in the sample.

In some embodiments, the Raman spectrum are analyzed using a machine learning algorithm (e.g., random forest (RF) and/or support vector machines (SVM)). In some embodiments, random forest training is used to construct a neural network decision tree. In some embodiments, spectrum are preprocessed prior to analysis (e.g., using background subtraction and vector normalization).

In some embodiments, the Raman spectrometer is a portable Raman spectrometer. In some embodiments, the Raman spectrometer is battery operated or AC operated. In some embodiments, the Raman spectrometer comprises a plurality of filters that filter the spectral band of the spectrometer to specific wavelengths or wavelength bands of light. In some embodiments, the Raman spectrometer is automated (e.g., including obtaining spectrum and analyzing data). In some embodiments, the Raman spectrometer performs spectroscopy and analysis in 5 minutes (e.g., 5, 4, 3, 2, or 1 minute) or less. In some embodiments, the method is performed at the point of care (e.g., at a veterinary clinic).

Further embodiments provide a method of treating a canine subject for a UTI, comprising: detecting the presence of a UTI using a method as described herein; and administering an antibiotic to the canine. In some embodiments, the method is repeated one or more times by retesting a new sample after administration of the antibiotic to the canine (e.g., to monitor treatment). In some embodiments, one or more antibiotics are selected based on the identity of the infectious disease agent identified.

Additional embodiments are described herein.

### DESCRIPTION OF THE FIGURES

The patent or application file contains at least one drawing executed in color. Copies of this patent or patent application publication with color drawings will be provided by the Office upon request and payment of the necessary fee.

FIG. 1 shows a schematic of an exemplary machine learning strategy utilized herein.

FIG. 2A-C shows a summary of random forest analysis of Raman spectrum that can be used to weight deep neural network analysis.

FIG. 3 shows exemplary sample filtration methods.

FIG. 4 shows classification of bacteria genus/species using machine learning analysis.

FIG. 5A-C shows confusion matrices for the data shown in FIG. 4.

FIG. 6A-E shows results of filtration of urine samples prior to analysis.

FIG. 7 shows classification of crystals from spiked and natural clinical canine urine samples.

FIG. 8A-E shows confusion matrices for the data shown in FIG. 7.

FIG. 9 shows classification of natural clinical canine urine samples containing white blood cells (Healthy Canine Urine (HCU) vs naturally occurring WBCs (NWBC)).

### DEFINITIONS

As used herein, the terms “detect”, “detecting”, or “detection” may describe either the general act of discovering or discerning or the specific observation of a composition.

The term “sample” as used herein is used in its broadest sense. In one sense it can refer to a biological sample. Biological samples may be obtained from animals (e.g., mammals, including companion animals such as canines, felines, and the like) and encompass fluids, solids, tissues, and gases. Biological samples include, but are not limited to urine and blood products, such as plasma, serum and the like. These examples are not to be construed as limiting the sample types applicable to the present disclosure.

As used herein, the term “dilution” refers to the process of reducing the concentration of a sample in solution. In some embodiments, samples (e.g., urine samples) are diluted by adding a solvent (e.g., water). For example, a 50% dilution of a 10 ml sample is 10 ml of the sample and 5 ml of solvent. In some of the examples described herein, a dilution of a sample is performed by removing a portion of the sample and replacing the sample with a solvent. For example, if 5 ml of a 10 ml sample is removed and replaced with solvent, the resulting sample is 50% sample and 50% solvent, or a 100% dilution of the original sample. In some embodiment, dilution is reported as a percentage of the original sample present in the diluted sample, rather than the percent dilution. For example, an 80% sample refers to a sample that is 80% sample and 20% solvent (e.g., 8 ml of sample and 2 ml of solvent).

### DETAILED DESCRIPTION OF THE DISCLOSURE

Provided herein are compositions and methods for diagnosing urinary tract infections. In particular, provided herein are compositions and methods for preparing canine urine samples and performing Raman spectroscopy detection of urinary tract infections in the samples. The present disclosure provides rapid, point of care analysis of urine samples that overcomes limitations of existing molecular, imaging, and Raman technologies.

By combining multi-omic detection with rapid, portable or small bench top instruments, and integrated data analysis components, the methods of the present disclosure provide both diagnostic information (e.g., the presence of a UTI), and further information regarding the genus, species, or strain of bacteria present, leukocyte identification, and the type of any crystals that are present. This results in improved antibiotic stewardship by veterinarians due to genus and species-level detection at the time of an initial patient visit.

Previous molecular assays for UTIs utilized PCR technology, which quantifies DNA and RNA, and is best-suited for pathogen detection in veterinary medicine in the reference lab setting. While there are some point of care PCR platforms, they have significant limitations in workflow that require technical skill sets above those commonly found in a veterinary clinic. PCR technologies can only target biological targets, not disease indicators such as crystals. PCR also cannot adequately identify host white blood cells (WBCs) due to additional host DNA contributions to urine samples. PCR technology is currently only offered at reference labs in veterinary medicine, takes 1-3 days to get results, and has limitations on multiplexing and automation abilities for POC applications. Another flaw associated with result interpretation in PCR is dead bacteria, which can produce false positives.

Urine sediment analysis is the evaluation of urine using a microscope in both automated (e.g., use of a high-powered camera with advanced imaging software) and non-automated environments (e.g., a veterinary technician). Urine sediment analysis is used to identify cells or crystals, based on shape detection, that could be indicative of disease or infection. Non-automated urine sediment analysis allows technicians to add urine sediment to a microscope slide and examine the slide to identify bacterial cells, crystals, or white blood cells via microscopy. This is a highly subjective process that results in human error and a high degree of variability. Automated urine sediment analysis utilizes a high-powered camera and identifies bacterial cells, white blood cells, and crystals using image recognition software. When identifying bacteria, however, genus, species and strain level detection of bacteria is not possible. Identification is facilitated by shape-based analyses. Even though the analysis is performed digitally, automated urine sediment analysis requires parallel manual analysis by technicians or veterinarians as results are often inconclusive and require 2-3 runs if the urine concentration is too low or too high. Due to this, non-automated urine sediment analysis is typically needed in conjunction.

Bacteria culture is a process that takes a patient sample (e.g., urine) and adds to material made for bacterial growth. After a few days (typically 2-5 days), the culture is ready for microscopic analysis and identification of bacteria by a technician. Bacteria culture requires the veterinary clinic to wait 2-5 days for results. In-house culturing requires additional purchases of high-capital equipment (e.g., incubators) to keep bacteria at constant temperatures. The limitations of bacteria culture for efficient diagnoses and medical decision-making is well-documented in the literature.

Raman spectroscopy, discovered by physicist Sir C. V. Raman, has been utilized for decades in the research setting, but several limitations prevented clinical/diagnostic application of Raman spectroscopy. These limitations include lack of a Raman method to facilitate real-time, point of care application; lack of ability to miniaturize Raman devices without sacrificing performance, to have an acceptable footprint for point of care use (corresponds with much lower device cost); lack of automated Raman-based detection of targets in biological samples with no user analysis or interpretation required; rapid bacterial genus, species and strain level detection at the point of care; rapid genus, species and strain level detection multiplexing at the point of care; rapid UTI detection techniques that do not rely on bacterial culture or amplification; the need for rapid UTI detection based on multi-omic information at the point of care, not just molecular vs protein vs cellular detection; the need for UTI detection technology at the point of care that does not require time-consuming urine processing (sample prep) protocols; and improved antibiotic stewardship practices at the point of care based on the ability to identify bacteria genus, species and strains in real time.

A number of Raman spectroscopy techniques, for example, Raman microspectroscopy, selective-sampling Raman microspectroscopy, coherent anti-Stokes Raman spectroscopy (CARS), surface enhanced Raman spectroscopy (SERS), fiber-optic Raman probes, and resonance Raman scattering (RRS) have been developed. However, each of these techniques have limitations that make them inadequate for use in the point of care setting.

Accordingly, provided herein is a sample preparation and analysis method that reduces background and utilizes portable, automated Raman spectroscopy suitable for point of care UTI detection, treatment and management.

In some embodiments, the present disclosure utilizes sample preparation methods that reduce or eliminate background signal in Raman spectroscopy caused by components of urine. For example, in some embodiments, a urine sample (e.g., from a canine) is diluted with a solvent (e.g., water such filtered water, sterilized water, tap water, distilled water, or reverse osmosis treated water). In some embodiments, the urine sample is diluted with water (e.g., with the addition of 10%, 20%, 30%, 40%, 50%, 60%, 70%, 80%, 90% or 100% the original volume of sample). In some embodiments, a portion of the urine sample is removed and replaced with water (e.g., 10%, 20%, 30%, 40%, 50%, 60%, 70%, 80%, 90% or 100% of the original volume of sample is removed and replaced with an equal or non-equal amount of water). In some embodiments, the sample is centrifuged (e.g., for 1, 2, 5, 10, 20, or 30 minutes) prior to removal of the supernatant. In some embodiments, the dilution of the sample reduces or eliminates background Raman signal (e.g., from urine) while still allowing detection of bacterial and/or crystal in the urine using Raman spectroscopy. In some embodiments, the sample is filtered. In some embodiments, sample preparation comprises a filtration step. Examples of suitable filtration material include but are not limited to, a polycarbonate membrane, a mixed cellulose ester, polyvinylidene difluoride, nylon, or cellulose acetate. In some embodiments, filtered samples are diluted with water, replacing centrifugation.

After sample preparation, samples are analyzed using Raman spectroscopy. The present disclosure is not limited to particular Raman spectrometers. In some embodiments, commercially available Raman spectroscopy systems are utilized. (See also U.S. Pat. No. 10,253,346, and U.S. patent application Ser. No. 16/451,901; each of which is herein incorporated by reference in its entirety).

In some embodiments, Raman spectrometers for use in the described methods are portable (e.g., light weight, table top instruments). In some embodiments, portability is enhanced by powering the instrument with a disposable or rechargeable battery. In some embodiments, instruments run on AC. In some embodiments, Raman spectrometers utilize filters to restrict spectrum to a single or narrow range of bandwidths.

In some embodiments, the Raman spectroscopy methods described herein utilize automated detection (e.g., generation of spectrum and analysis of spectrum). In some embodiments, algorithms and pathogen libraries are embedded in the system's onboard software to achieve automated analysis and output information.

In some embodiments, the Raman spectroscopy is rapid (e.g., less than 5, 4, 3, 2, or 1 minute, including or not including data analysis).

In some embodiments, the Raman spectroscopy methods described herein utilize multi-omic information to generate a molecular fingerprint indicative of disease. For example, in some embodiments, a single Raman spectrum comprises peaks related to the presence of one or more of proteins, nucleic acids (e.g., DNA and/or RNA), carbohydrates, and small molecules (e.g., metabolites).

In some embodiments, Raman spectroscopy methods described herein identify the presence of bacteria and/or crystals indicative of the presence of a UTI in a subject (e.g., a canine subject). For example, in some embodiments, Raman spectroscopy identifies the presence and/or genus, species, and strain of bacteria.

In some embodiments, the methods described herein detect the presence of crystals in urine samples. Normal dog urine is slightly acidic (pH 6-6.5) and contains metabolic waste products. Changes in urine's pH and abundance of certain metabolites (e.g., as a result of a UT) facilitates urine crystal formation. For example, growing bacteria produces urease enzyme, which breaks down urea to ammonia. Ammonia production turns urine alkaline resulting in Struvite crystal formation. Struvite crystals (STR) are comprised of magnesium ammonium phosphate. They are a natural constituent of dog's urine and remains dissolved in slightly acidic urine. Alkaline urine and higher concentrations of struvite induce crystallization of struvite, which can form bladder stones.

In addition, the abundance of calcium, citrate or oxalates or certain diets turns urine acidic, which facilitates calcium oxalate crystal formation. Some calcium oxalate crystals are comprised of calcium oxalate monohydrate (COM), which is a natural constituent of dog urine that crystalizes under higher concentrations and acidic pH. COM can form bladder and kidney stones. Calcium oxalate dehydrate (COD) crystals are also formed in urine when there is ethylene glycol or antifreeze poisoning.

In some embodiments, the presence of crystals is indicative of a UTI. In some embodiments, the presence of crystals is indicative of an underlying condition (e.g., a condition that predisposes a subject to UTIs or an unrelated medical condition). For example, in some embodiments, when the presence of crystals but not UTI is identified, an alternative intervention is recommended (e.g., therapeutic diet, nutritional supplements, increased fluid intake, and medical examinations for kidney stones and kidney disorders).

In some embodiments, the head (e.g., first 5, 10, 15, or 20% of the spectra), tail (e.g., last 5, 10, 15, or 20% of the spectra), LED peak, and urea peak are removed prior to analysis.

In some embodiments, the Raman spectroscopy systems described herein utilize a machine learning algorithm for data analysis. Machine learning algorithms build a mathematical model based on sample data, known as “training data”, in order to make predictions or decisions without being explicitly programmed to perform the task. In some embodiments, machine learning algorithms utilize support-vector machines and/or random forest algorithms and/or deep neural networks.

In machine learning, support-vector machines (SVMs, also support-vector networks; Cortes, Corinna; Vapnik, Vladimir N. (1995). Machine Learning. 20 (3): 273-297; herein incorporated by reference in its entirety) are supervised learning models with associated learning algorithms that analyze data used for classification and regression analysis. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall.

Random forests or random decision forests are an ensemble learning method for classification, regression and other tasks that operates by constructing a multitude of decision trees at training time and outputting the class that is the mode of the classes (classification) or mean prediction (regression) of the individual trees (See e.g., Ho, Tin Kam (1995) Proceedings of the 3rd International Conference on Document Analysis and Recognition, Montreal, QC, 14-16 Aug. 1995. pp. 278-282; Ho TK (1998) IEEE Transactions on Pattern Analysis and Machine Intelligence. 20 (8): 832-844; each of which is herein incorporated by reference in its entirety).

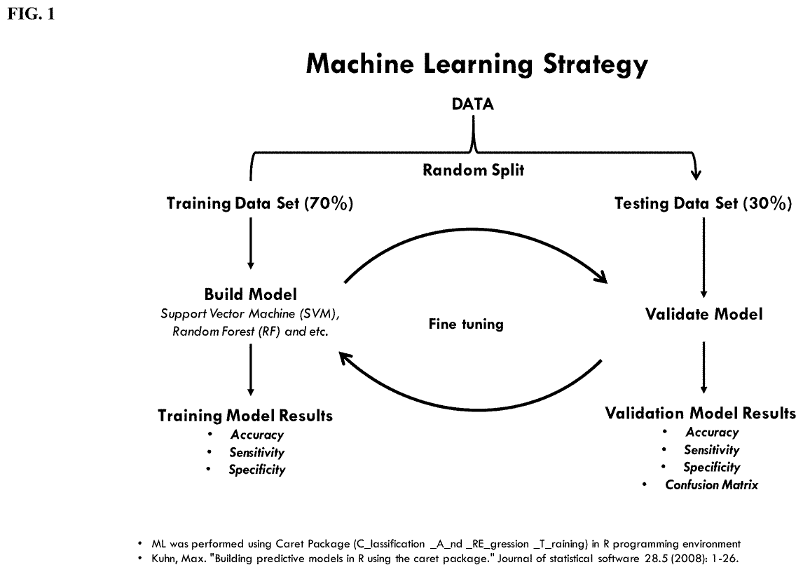

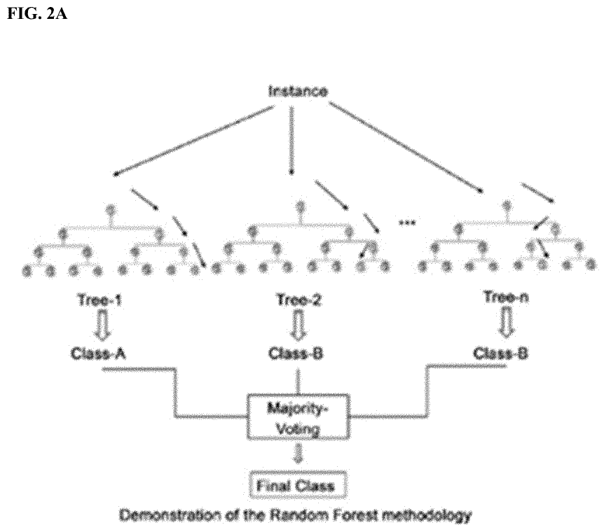

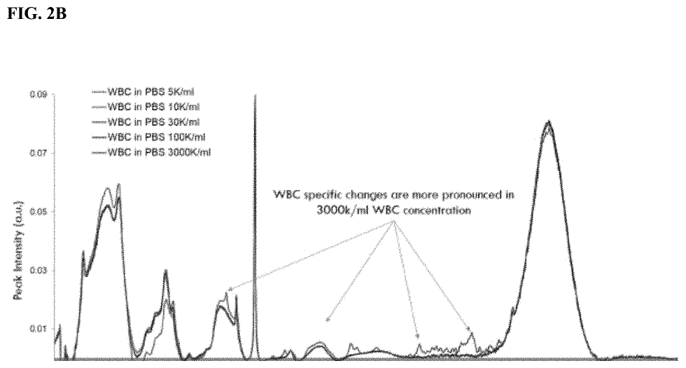

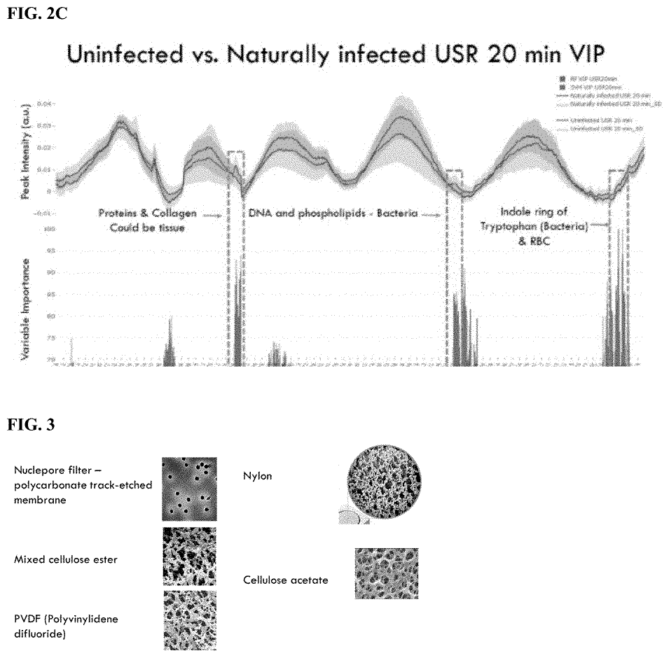

FIGS. 1-2 (Example 1) show an exemplary workflow for machine learning methods such as random forest analysis. Random forest training is used to construct a neural network decision tree in an iterative fashion with key focal nodes which center around specific areas of interest on the Raman spectra. Random forest and biology are used to figure out which questions to ask to most efficiently identify the pathogen's character. In other words, Random forest is used to decipher how to partition the matching library with a minimum depth tree (e.g., to prune the decision tree).

In some embodiments, machine learning methods include deep neural networks (DNN). A deep neural network (DNN) is an artificial neural network with multiple layers between the input and output layers. The DNN finds the correct mathematical manipulation to turn the input into the output, whether it be a linear relationship or a non-linear relationship (See e.g., Bengio, Yoshua (2009). “Learning Deep Architectures for AI” (PDF). Foundations and Trends in Machine Learning. 2 (1): 1-127 and Schmidhuber, J. (2015). “Deep Learning in Neural Networks: An Overview”. Neural Networks. 61: 85-117. arXiv:1404.7828; each of which is herein incorporated by reference in its entirety). Many different deep architectures exist which represent variants of a few basic approaches.

The methods of the present disclosure provide for rapid, point of care diagnosis, prognosis, and patient monitoring applications. Embodiments of the disclosure provide methods for determining a treatment course of action, administering a treatment, and/or monitoring a treatment. For example, in some embodiments, the results of the analysis methods described herein are used to select an initial antibiotic based on the type of bacteria and/or stones present in a sample. Following administration of the antibiotic to the subject, in some embodiments, analysis is repeated one or more times by retesting a new sample after administration of the antibiotic to the canine to determine the efficacy of the antibiotic. In some embodiments, based on the results of the analysis, the treatment is stopped (e.g., when no further symptoms of disease are identified) or altered (e.g., when symptoms of disease are still present after a treatment course). The analysis is repeated as many times as needed prior to, during, or after treatment. The below Table provides a list of exemplary antibiotics for use with specific UTI-causing bacteria (See e.g., Antimicrobial Therapy in Veterinary medicine, 5th edition, 2013; Vet Clin NA Small Anim Pract 28(2), 1998; each of which is herein incorporated by reference in its entirety).

| | | | | | | Antipseudomonal | Penicillinase/−lactamase- |
|---|---|---|---|---|---|---|---|
| | | | | | Aminopenicillins | penicillins | resistant penicillins |
| | | | Sulphonamides + | | (amoxicillin, | (carbenicillin, | (methicillin, |
| UTI | Canine | Therapeutics: | diaminopyrimidines | Penicillins | ampicillin. . .) | ticarcillin. . .) | cioxacillin) |
| 1 | E. coli | Recommended | X | | X | X | |
| | | First Line | | | | | |
| 2 | Enterococcus casts | Recommended | | | | | |
| | | First Line | | | | | |
| 3 | Gram− | Recommended | X | X | X | X | |
| | | First Line | | | | | |
| 4 | Gram+ | Recommended | X | X | X | | X |
| | | First Line | | | | | |
| 5 | Klebsiella | Recommended | X | | | | |
| | | First Line | | | | | |
| 6 | Lepto | Recommended | | | | | |
| | | First Line | | | | | |
| 7 | Proteus | Recommended | X | | | X | |
| | | First Line | | | | | |
| 8 | Pseudomonas | Recommended | | | | | |
| | | First Line | | | | | |
| 9 | Staph. | Recommended | X | X | | X | X |
| | | First Line | | | | | |
| 10 | Strep. | Recommended | X | X | X | X | X |
| | | First Line | | | | | |

| | | Group 1 | Group 2 - | Group 3 | | | |
|---|---|---|---|---|---|---|---|
| | | parenteral | Oral | parenteral | | | |
| | Potentiated | cephalosporins | cephalosporins: | cephalosporins: | Group 4 | Group 6 | |
| | Penicillins | (cephapirin, | 1st generation | 2nd generation | parenteral | parenteral | |
| | (amoxicillin + | cefazolin, | (cefadroxil, | (cefuroxime, | cephalosporins | cephalosporins: | |
| UTI | clavulanate) | cephalothin) | cephalexin) | cefoxitin) | (ceftiofur) | (ceftazidime) | Aminoglycosides |
| 1 | | | | X | X | X | |
| 2 | X | | | X | | | |
| 3 | | | | X | X | X | X |
| 4 | X | X | X | X | X | X | |
| 5 | | | | X | X | X | |
| 6 | | | | | | | X |
| 7 | | X | | X | X | X | X |
| 8 | X | | | X | | X | |
| 9 | X | X | X | | | | |
| 10 | X | X | X | | X | X | |

| UTI | Tetracyclines | Lincosamides | Macrolides | Fluoroquinolones | Phenicols | Nitroimidazoles |
|---|---|---|---|---|---|---|
| 1 | | | | | X | |
| 2 | | | | | X | |
| 3 | | | | X | X | |
| 4 | | X | X | | X | |
| 5 | | | | X | X | |
| 6 | X | X | X | | | |
| 7 | | | | X | X | |
| 8 | | | | | | |
| 9 | | | X | X | X | |
| 10 | | | X | | X | |

### EXPERIMENTAL

The following examples are provided in order to demonstrate and further illustrate certain preferred embodiments and aspects of the present disclosure and are not to be construed as limiting the scope thereof.

### Example 1

This example describes the use of a Machine learning (FIG. 1) and a neural network to analysis Raman spectra. Data was split based on the group 70% training and 30% testing data sets. Random Forest and Support Vector Machine were used for spectral analysis. Models were optimized on training data set and their results were validated by testing data sets. Select Raman spectral regions were eliminated from analysis due to scattering, LED peaks and urea peaks.

Random Forest training is used to construct a neural network decision tree in an iterative fashion with key focal nodes which center around specific areas of interest on the Raman spectra. Branches are created and iterated based upon the amount of information gained (entropy) by the resulting node (FIG. 2A). Random Forest allows one to have an in-depth analysis with relatively small amounts of data.

Random Forest iterations are used to identify important biological differentiation factors between spectra in a library. These important differentiators, such as specific peaks of interest (FIG. 2B), are then given a special weight in the DNN algorithms.

With the learned information about distinguishing peaks, the DNN can ‘skip’ or place less weight on less important nodes and focus its computing efforts on pivotal nodes which yield a more accurate, efficient, and consistent result (FIG. 2C).

### Example 2

This example describes the classification of bacteria genus/species using Raman spectroscopy of natural clinical canine urine samples (Uninfected vs Naturally Infected (UN)). Bacteria levels varied between 0.3 to 1 optical density (OD) in water and in contrived canine urine samples spiked with bacteria. Natural clinical samples from infected dogs were validated for the type of bacteria/colony forming units (CFU) per mL by an independent lab analysis.

Data was obtained in 100% urine, 80% water-20% urine, and 90% water-10% urine. Data is compared with that in pure water (no contact with urine) or uninfected urine.

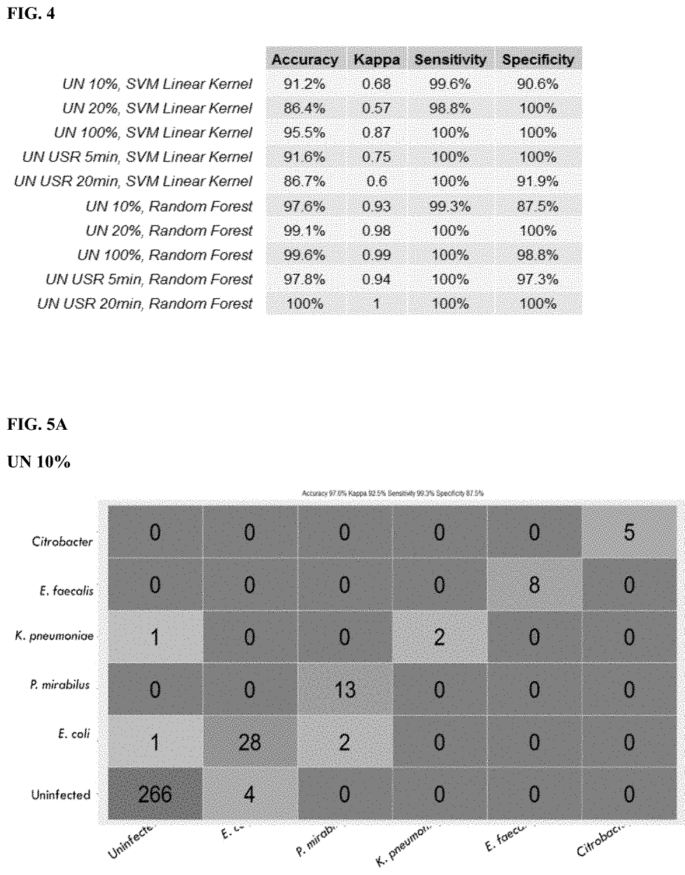

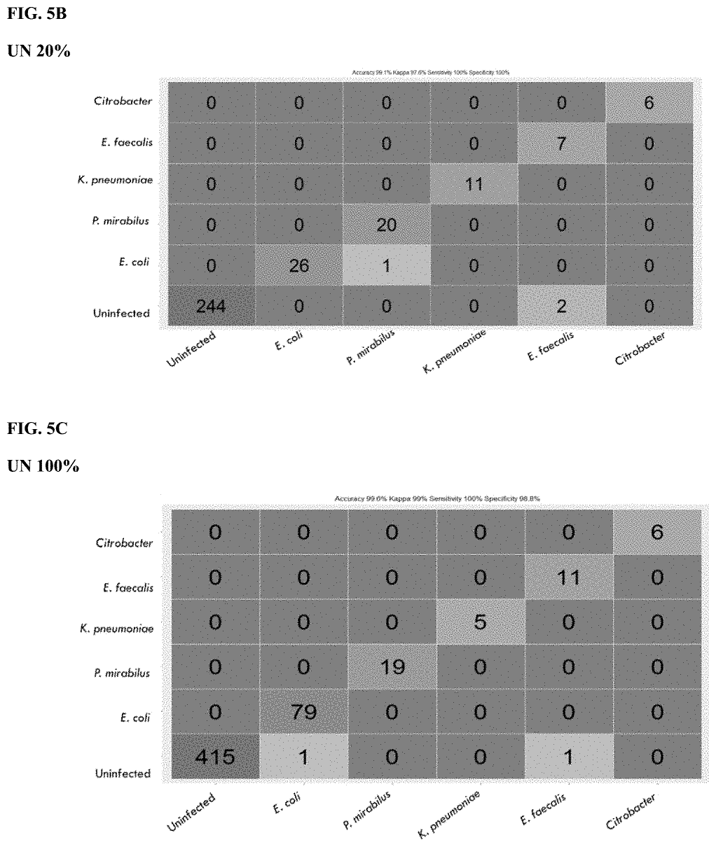

Table 1 shows classification of gram positive and negative samples in pure water vs spiked water, uninfected urine vs spiked urine, and uninfected vs naturally infected urine. Gram classification accuracy of >95% was observed in uninfected vs spiked urine and >97% in water vs spiked water and uninfected vs naturally infected urine. FIGS. 4 and 5 show classification of bacteria genus/species and corresponding Confusion Matrices for random forest analysis. Confusion Matrices indicate the classification predicted by the machine learning algorithm compared to the actual classification. FIG. 5 shows the Confusion Matrices for random forest analysis of 10%, 20%, and 100% urine. All sample preparation methodologies and algorithms show the ability to classify bacterial infections by genus/species. Classification accuracy and kappa value show slight improvement with an increasing concentration of urine with both SVM and random forest algorithms.

TABLE 1

| | Non-Clinical^{2, 3} | | Contrived Clinical^{1, 2, 3} | | Natural Clinical³ | |
|---|---|---|---|---|---|---|
| | (Spiked Water) | | (Spiked Urine) | | (Urine) | |
| | | No of | | No of | | No of |
| | Accuracy⁴ | tests⁶ | Accuracy⁴ | tests⁶ | Accuracy⁵ | tests⁶ |
| Gram | 97.7% | 226 (10,410) | 95.2% | 24 (425) | 97.8% | 1 (25) |
| Positive | | | | | | |
| Gram | 97.8% | 333 (19,595) | 95.2% | 41 (880) | 100% | 9 (225) |
| Negative | | | | | | |

### Example 3

This example describes the use of filtration methods of urine sample prior to analysis with Raman Spectroscopy. Bacteria filtration was evaluated as a less labor-intensive alternative to centrifugation. A syringe filter back flush methodology was used to compare:
Mix cellulose ester syringe filters (0.2, 0.4 & 0.8 micron)
Polycarbonate syringe filters (0.2, 0.4 & 0.8 micron)
Nylon syringe filters (0.2, 0.4 & 0.8 micron)
Polyvinylidene Fluoride (PVDF) syringe filters (0.2, 0.4 & 0.8 micron)
   The OD results of urine, urine spiked with bacteria and recovered bacteria/materials after filtration were plotted.

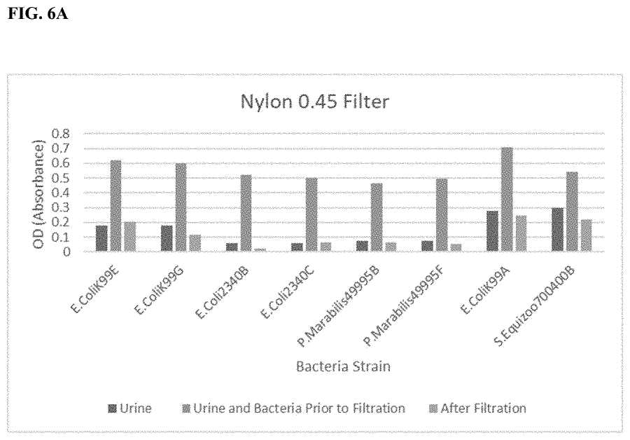

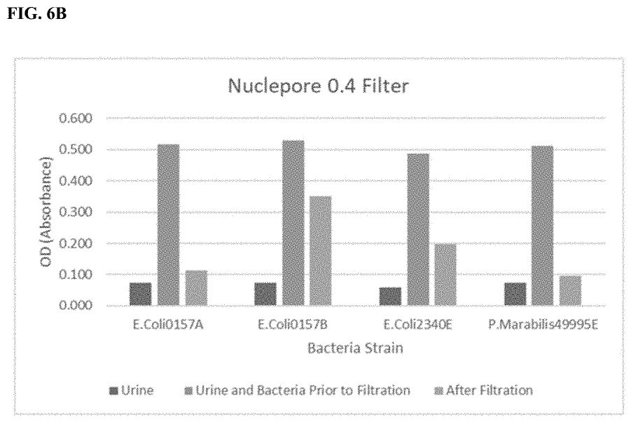

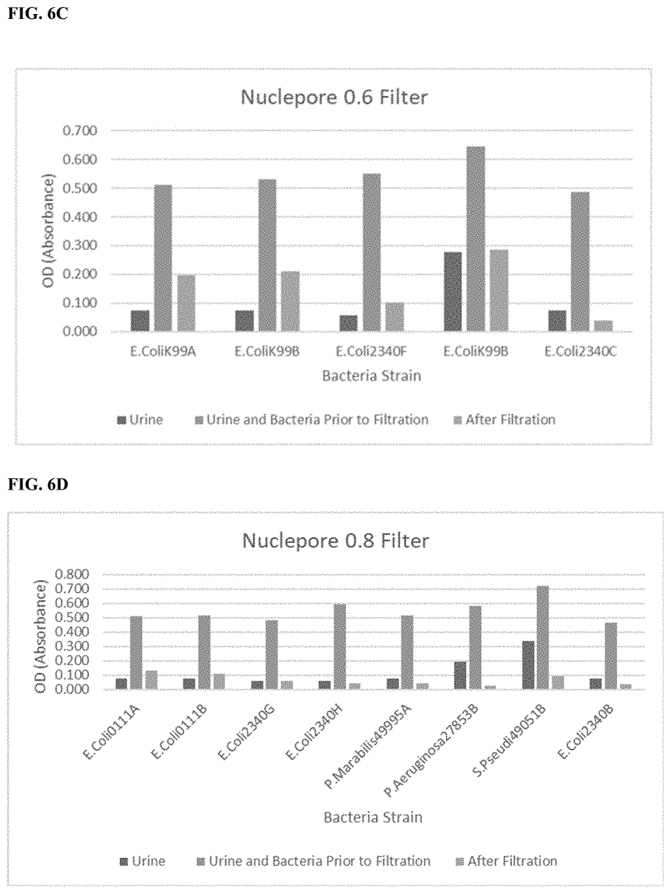

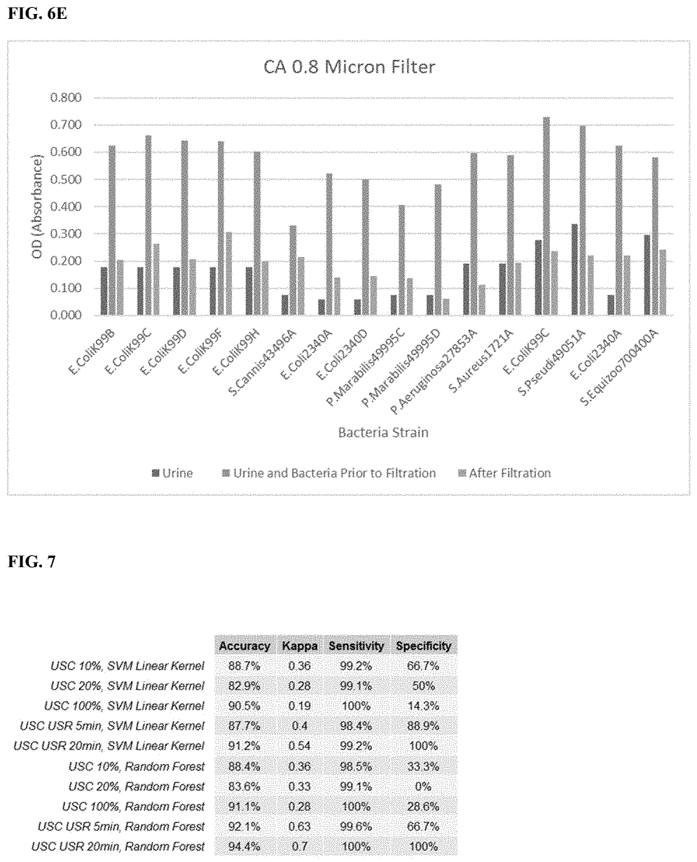

FIG. 3 shows the different filtration methods used. Filtration of urine using any of the filtering options tested resulted in a reduction of background fluorescence in Raman spectra as compared to unfiltered urine samples. Different types of filters demonstrated different levels of bacteria recovery. FIG. 6 show the results.

### Example 4

This example describes classification of crystals using Raman spectroscopy.

Crystals were obtained through sectioning bladder and kidney stone and infrared spectroscopy analysis. The types of spiked crystals included:

Struvite Crystals (STR): comprised of magnesium ammonium phosphate. Struvite is a natural constituent of dog's urine and remains dissolved in slightly acidic urine. Alkaline urine and higher conc. of Struvite induces crystallization of Struvite resulting in bladder stones.

Calcium Oxalate Crystals: There are two types of Calcium oxalate crystals. Calcium Oxalate Monohydrate (COM) is a natural constituent of dog urine. It crystalizes under higher concentrations and acidic pH and forms bladder and kidney stones. Calcium Oxalate Dehydrate (COD) crystals form in urine when there is ethylene glycol or antifreeze poisoning.

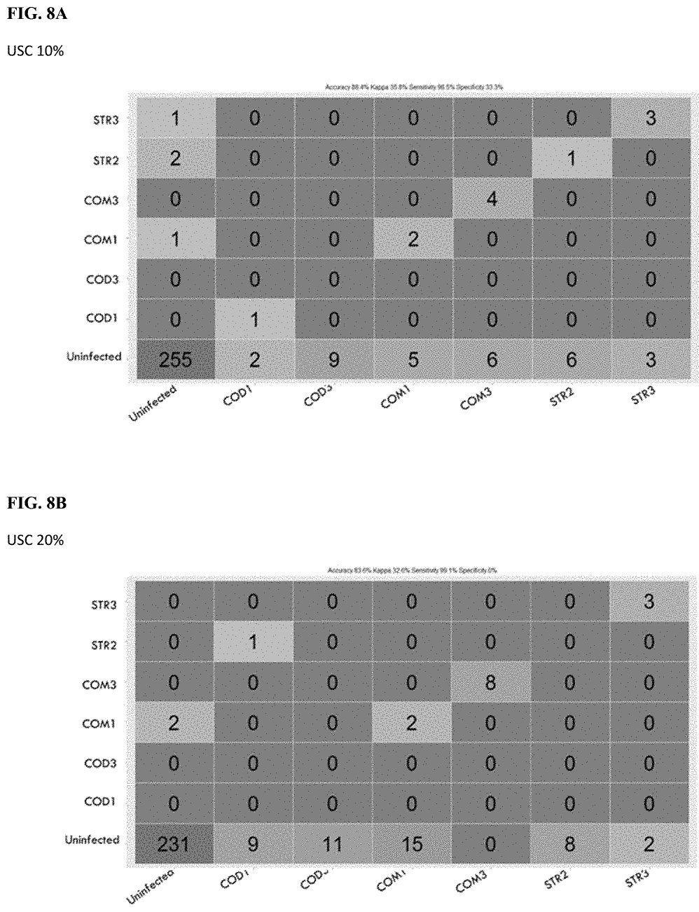

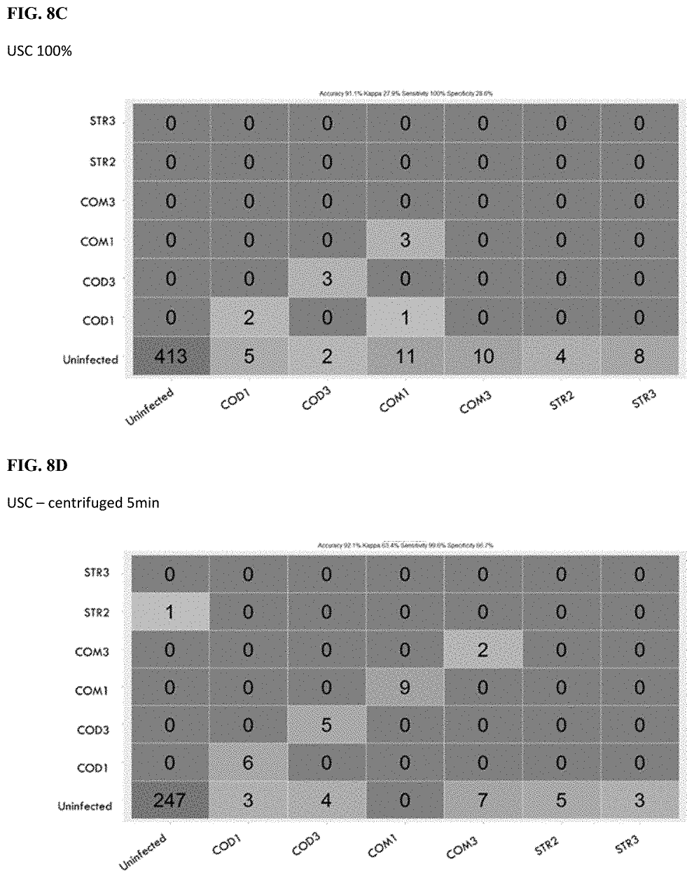

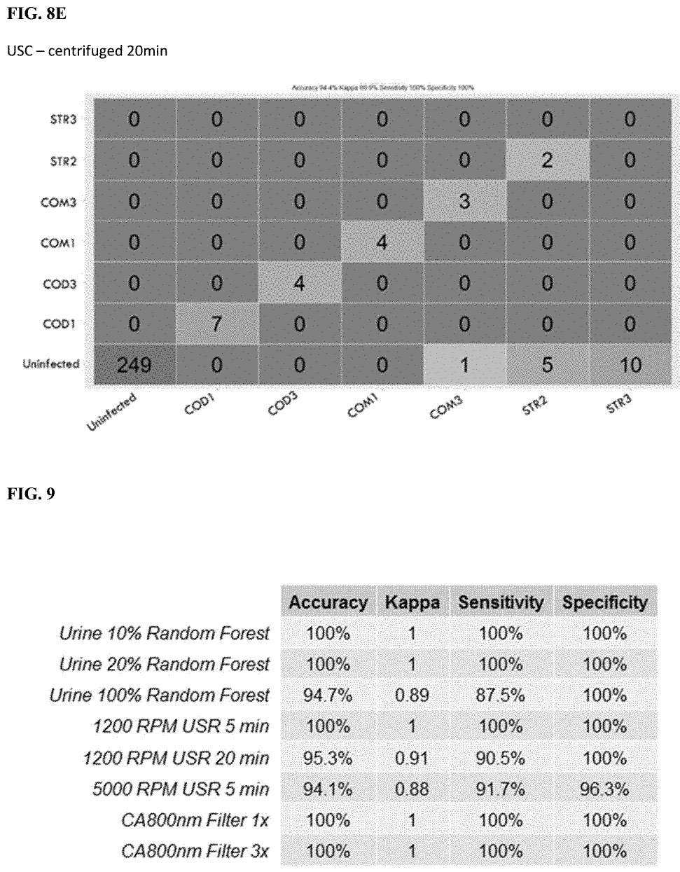

Results are shown in FIGS. 7 and 8. All sample preparation methods and algorithms tested show the ability to classify spiked crystals in urine compared to uninfected urine. Crystal classification accuracy was impacted by dilution and centrifugation techniques.

### Example 5

This Example describes classification of natural clinical canine urine samples containing white blood cells (Healthy Canine Urine (HCU) vs naturally occurring WBCs (NWBC)). Concentrations of WBCs in natural clinical dog samples were determined by independent lab analysis. Sample were prepared by urine dilution, USR at different centrifugation speeds, or filtering. Spectral analysis was performed using a random forest algorithm. Results are shown in FIG. 9. All sample preparation methods show the ability to classify white blood cell containing urine compared to uninfected urine. White blood cell classification accuracy was impacted by various dilution, filtration and centrifugation techniques.

All publications, patents and patent applications mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. A method of identifying the presence of a urinary tract infection (UTI) in a urine sample from a canine, comprising:
a) diluting said urine sample with water to generate a diluted sample; and
b) obtaining a Raman spectrum of said diluted sample using a Raman spectrometer, wherein said Raman spectrum identifies the presence of a UTI in said sample.

2. The method of claim 1, wherein said water is purified or unpurified water.

3. The method of claim 1, wherein said diluted sample is diluted 20%.

4. The method of claim 1, wherein said diluted sample is diluted 50%.

5. The method of claim 1, wherein said diluted sample is diluted 75%.

6. The method of claim 1, wherein said diluting step comprises i) centrifuging said urine sample to generate a pellet and a supernatant; and ii) removing at least a portion of said supernatant and replacing it with water to generate a diluted sample.

7. The method of claim 6, wherein 20% of said supernatant is replaced with water.

8. The method of claim 6, wherein 50% of said supernatant is replaced with water.

9. The method of claim 6, wherein 100% of said supernatant is replaced with water.

10. The method of claim 1, wherein said presence of a UTI is determined based on the presence of bacteria and/or crystals in said urine.

11. The method of claim 1, wherein said sample is filtered prior to said obtaining a Raman spectrum.

12. The method of claim 11, wherein said sample is filtered with a material selected from the group consisting of a polycarbonate membrane, a mixed cellulose ester, polyvinylidene difluoride, nylon, and cellulose acetate.

13. The method of claim 1, wherein said diluted sample exhibits reduced or no background interference from urine.

14. The method of claim 1, wherein said Raman spectroscopy generates a molecular fingerprint of said sample.

15. The method of claim 1, wherein said Raman spectrum are analyzed using a machine learning algorithm.

16. The method of claim 15, wherein said machine learning algorithm comprises the use of random forest training to construct a neural network decision tree.

17. The method of claim 1, wherein said Raman spectrometer comprises a plurality of filters that filter the spectral band of said spectrometer to specific wavelengths of light.

18. The method of claim 1, wherein said Raman spectrometer is automated.

19. The method of claim 1, wherein said method is performed at the point of care.

20. A method of treating a UTI in a canine subject, comprising:
a) detecting the presence of a UTI in a sample from said canine subject using a method of claim 1; and
b) administering an antibiotic to said canine subject.
